(19) United States
(12) Patent Application Publication
(10) Pub. No.: US 2014/0276064 A1
(43) Pub. Date: Sep. 18, 2014

(54) ULTRASOUND PROBE AND ULTRASOUND DIAGNOSIS APPARATUS

(71) Applicants: Kabushiki Kaisha Toshiba, Minato-ku (JP); Toshiba Medical Systems Corporation, Otawara-shi (JP)

(72) Inventors: Takashi KUBOTA, Otawara-shi (JP); Takashi TAKEUCHI, Otawara-shi (JP); Yutaka OONUKI, Otawara-shi (JP); Yasuhisa MAKITA, Nasushiobara-shi (JP)

(73) Assignees: Kabushiki Kaisha Toshiba, Minato-ku (JP); Toshiba Medical Systems Corporation, Otawara-shi (JP)

(21) Appl. No.: 14/289,060

(22) Filed: May 28, 2014

Related U.S. Application Data
(63) Continuation of application No. PCT/JP2013/072473, filed on Aug. 22, 2013.

(30) Foreign Application Priority Data
Aug. 30, 2012 (JP) 2012-190672
Aug. 21, 2013 (JP) 2013-171471

(51) Int. Cl. A61B 8/00; A61B 8/08; A61B 8/14

(52) U.S. Cl. CPC A61B 8/4461; A61B 8/4494; A61B 8/145; A61B 8/5207; A61B 8/461
USPC 600/444; 600/459

(57) ABSTRACT

The object of the present invention is to provide a ultrasound probe and an ultrasound diagnosis apparatus which are able to reduce the time and labor for the operation and to prevent image quality from deteriorating. the ultrasound probe of the embodiments comprises an acoustic window, a transducer part, and a moving mechanism. The acoustic window comprises more than one holding surface with a recessed surface shape that engages with a subject. The transducer part comprises a plurality of transducers arranged in one or a plurality of rows for transceiving ultrasound waves with respect to the subject that comes into contact with the holding surface via the acoustic window. The moving mechanism moves the transducer part in a direction vertical to the arrangement direction of each row of the transducers.

### CROSS-REFERENCE TO RELATED APPLICATIONS

This application is based upon and claims the benefit of priority from Japanese Patent Applications No. 2012-190672, filed Aug. 30, 2012 and Japanese Patent Applications No. 2013-171471, filed Aug. 21, 2013; the entire contents of all of which are incorporated herein by reference.

### FIELD

The embodiments of the present invention relate to an ultrasound probe and an ultrasound diagnosis apparatus for diagnosis by imaging the internal body of a subject using ultrasound waves.

### BACKGROUND

Ultrasound diagnosis apparatuses for examining a subject by transmitting ultrasound waves into the subject to use reflected waves from the inside of the subject are widely utilized in the medical field. The ultrasound diagnosis apparatus includes an ultrasound probe for transceiving ultrasound waves with respect to the subject by being contacted thereto, and an apparatus main body for generating image data based on reception signals obtained by driving the ultrasound probe.

Incidentally, some ultrasound probes include a transducer part having a plurality of transducers arranged in a one-dimensional array form and a moving mechanism for moving this transducer part. Jelly to be interposed on the surface contacting the subject or an elastic water bag to be abutted on the subject is used in order to propagate ultrasound waves from the ultrasound probe to the subject.

However, in the case that the subject is a part that includes a curved surface, a plenty of jelly is required which is problematic in that both applying and wiping off the jelly take time and labor. Furthermore, deteriorating image quality of image data, as a result of the subject moving when abutting the water bag, is also problematic.

The embodiments have been conducted intended to solve the abovementioned problematic points, with the purpose of providing an ultrasound probe and an ultrasound diagnosis apparatus capable of reducing the time and labor for the operation and preventing image quality from deteriorating.

### BRIEF DESCRIPTION OF THE DRAWINGS

FIG. 1A It is a plane drawing depicting the configuration of an ultrasound diagnosis apparatus according to a first embodiment.

FIG. 1B It is a front view depicting the configuration of the ultrasound diagnosis apparatus according to the first embodiment.

FIG. 1C It is a side view depicting the configuration of the ultrasound diagnosis apparatus according to the first embodiment.

FIG. 2 It is a block diagram depicting the configuration of an ultrasound probe and an apparatus main body according to the first embodiment.

FIG. 3A It is a plane drawing depicting one example of the configuration details of the ultrasound probe according to the first embodiment.

FIG. 3B It is a cross-sectional view taken along arrows A-A of FIG. 3A.

FIG. 3C It is a cross-sectional view taken along arrows B-B of FIG. 3A.

FIG. 4A It is a plane drawing depicting another configuration example of the ultrasound probe according to the first embodiment.

FIG. 4B It is a cross-sectional view taken along arrows A-A of FIG. 4A.

FIG. 4C It is a cross-sectional view taken along arrows B-B of FIG. 4A.

FIG. 5 It is a drawing depicting one example of screens displaying data for each image on a display according to the first embodiment.

FIG. 6A It is a plane drawing depicting the configuration of an ultrasound probe according to a second embodiment.

FIG. 6B It is a cross-sectional view taken along arrows A-A of FIG. 6A.

FIG. 6C It is a cross-sectional view taken along arrows B-B of FIG. 6A.

FIG. 7A It is a plane drawing depicting the configuration of an ultrasound probe according to a third embodiment.

FIG. 7B It is a cross-sectional view taken along arrows A-A of FIG. 7A.

FIG. 7C It is a cross-sectional view taken along arrows B-B of FIG. 7A.

FIG. 8 It is a cross-sectional view taken along arrows C-C of FIG. 7C.

FIG. 9 It is a plane drawing depicting the configuration of an ultrasound probe according to a fourth embodiment.

FIG. 10A It is a cross-sectional view taken along arrows A-A of FIG. 9.

FIG. 10B It is a cross-sectional view taken along arrows B-B of FIG. 9.

FIG. 11A It is a plane drawing depicting the configuration of an ultrasound probe according to a fifth embodiment.

FIG. 11B It is a cross-sectional view taken along arrows A-A of FIG. 11A.

FIG. 11C It is a cross-sectional view taken along arrows B-B of FIG. 11A.

FIG. 12A It is a plane drawing depicting the configuration of an ultrasound probe according to a sixth embodiment.

FIG. 12B It is a cross-sectional view taken along arrows A-A of FIG. 12A.

FIG. 12C It is a cross-sectional view taken along arrows B-B of FIG. 12A.

FIG. 13A It is a plane drawing depicting the configuration of an ultrasound probe according to a seventh embodiment.

FIG. 13B It is a cross-sectional view taken along arrows A-A of FIG. 13A.

FIG. 13C It is a cross-sectional view taken along arrows B-B of FIG. 13A.

FIG. 14A It is a plane drawing depicting the configuration of an ultrasound probe according to an eighth embodiment.

FIG. 14B It is a cross-sectional view taken along arrows A-A of FIG. 14A.

FIG. 14C It is a cross-sectional view taken along arrows B-B of FIG. 14A.

FIG. 15A It is a plane drawing depicting the configuration of an ultrasound probe according to a ninth embodiment.

FIG. 15B It is a cross-sectional view taken along arrows A-A of FIG. 15A.

FIG. 15C It is a cross-sectional view taken along arrows B-B of FIG. 15A.

### DETAILED DESCRIPTION

To achieve the above objective, the ultrasound probe of the embodiments comprises an acoustic window, a transducer part, and a moving mechanism. The acoustic window comprises more than one holding surface with a recessed surface shape that engages with a subject. The transducer part comprises a plurality of transducers arranged in one or a plurality of rows for transceiving ultrasound waves with respect to the subject that comes into contact with the holding surface via the acoustic window. The moving mechanism moves the transducer part in a direction vertical to the arrangement direction of each row of the transducers.

Hereinafter, the embodiments are described with reference to the drawings.

### First Embodiment

FIGS. 1A to 1C are exterior drawings depicting the configuration of an ultrasound diagnosis apparatus according to a first embodiment. FIG. 1A shows a plane drawing of the ultrasound diagnosis apparatus. FIG. 1B shows a front view of the ultrasound diagnosis apparatus. Moreover, FIG. 1C shows a side view of the ultrasound diagnosis apparatus. This ultrasound diagnosis apparatus 100 includes an ultrasound probe 10 for transceiving ultrasound waves with respect to a subject P and an apparatus main body 70 for generating image data based on reception signals obtained by driving the ultrasound probe 10.

FIG. 2 is a block diagram depicting the configuration of the ultrasound probe 10 and the apparatus main body 70. The ultrasound probe 10 includes a transducer part 20 having a plurality of transducers arranged in one row for transceiving ultrasound waves with respect to the subject P, and a moving mechanism 60 for moving the transducer part 20 in a predetermined direction.

The transducer part 20 transmits ultrasound waves into the subject P at a position moved by the moving mechanism 60 by a drive signal from a transmitter 81 in a transceiver 80 of the apparatus main body 70, and receives reflected waves resulting from transmitting ultrasound waves from the inside of the subject P to convert the received waves to reception signals.

The apparatus main body 70 includes the transceiver 80 for processing reception signals obtained from the drive of the transducer part 20 in the ultrasound probe 10 and obtained from the transducer part 20 by this drive, a signal processor 90 for generating data of imaging region from the signal processed by the transceiver 80, and an image data generator 91 for generating image data based on the data generated by the signal processor 90.

Further, the apparatus main body 70 includes a display 92 for displaying the image data generated by the image data generator 91, an operation part 98 for conducting input, and the like, of each command signal, and a system controller 99. The system controller 99 controls each unit of the moving mechanism 60 of the ultrasound probe 10, the transceiver 80, the signal processor 90, the image data generator 91, and the display 92.

The transceiver 80 includes a transmitter 81 that generates drive signals for driving the transducer part 20, and a receiver 82 for conducting phasing and addition with respect to the reception signals obtained from the transducer part 20. The transceiver 80 conducts ultrasound scanning in the arrangement direction of the transducers of the transducer part 20 based on imaging conditions such as gain, dynamic range, transmitting frequency, pulse repetition frequency, visual field depth, visual field angle, and frame rate supplied from the system controller 99.

The transmitter 81 generates a rate pulse to determine a repetition cycle (Tr) of the ultrasound pulse to radiate to the subject P. The transmitter 81 then provides, to the rate pulse, a delay time for convergence to converge ultrasound beams at a predetermined depth in each depth direction inside the subject P and a delay time for deflection to scan in each depth direction by transmitting waves. The transmitter 81 generates a drive pulse, which is a drive signal, from the rate pulse.

The receiver 82 secures a sufficient S/N by amplifying minute reception signals obtained from the transducer part 20. The receiver 82 then provides, to the reception signals, a delay time for convergence to converge ultrasound beams from a predetermined depth in each depth direction inside the subject P and a delay time for deflection to set a directionality of the ultrasound beams in the depth direction. The receiver 82 performs the phasing and addition on the reception signals to generate one signal, and then outputs the signal to the signal processor 90.

The signal processor 90 performs envelop detection on the reception signal, which has been subjected to the phasing and addition processing, from the receiver 82 of the transceiver 80, and then performs logarithmic transformation thereon. Further, the signal processor 90 converts the logarithmically transformed signal to a digital signal to generate data of the imaging region that has been scanned with ultrasound waves. The image data generator 91 performs scanning conversion on each data output from the signal processor 90 for displaying images. The image data generator 91 generates two-dimensional image data in which the imaging region has been imaged by scanning the inside of subject P with ultrasound waves. The image data generator 91 also generates three-dimensional image data from the two-dimensional image data that has been generated at a plurality of positions by moving the transducer part 20. Furthermore, the display 92 includes, for example, a liquid crystal panel, and displays the image data that has been generated by the image data generator 91.

The operation part 98 includes input devices, such as switches, whereby, for example, an ID for identifying the subject P is input by operating the input device. The operation part 98 also conducts inputs for setting the imaging conditions, such as gain, dynamic range, transmitting frequency, pulse repetition frequency, visual field depth, visual field angle, and frame rate. Furthermore, the system controller 99 includes a CPU as well as a memory circuit and comprehensively controls the moving mechanism 60 of the ultrasound probe 10, the transceiver 80, the signal processor 90, the image data generator 91, and the display 92, based on the input information entered through the operation part 98.

Next, the configuration of the ultrasound probe 10 is described into the details.

FIGS. 3A to 3C are drawings depicting one example of the configuration details of the ultrasound probe 10. FIG. 3A shows a plane drawing of the ultrasound probe 10. Further, FIG. 3B shows a cross-sectional view taken along arrows A-A of FIG. 3A while FIG. 3C shows a cross-sectional view taken along arrows B-B of FIG. 3A. The ultrasound probe 10 includes an acoustic window 40 that comes into contact with the subject P, the transducer part 20, the moving mechanism 60, and a casing 65 for holding the moving mechanism 60 and the acoustic window 40.

The acoustic window 40 is excellent in properties of propagating ultrasound waves, and made from, for example, a hard plastic material whereby a curved surface is easily formed without deformation, and has more than one holding surfaces in a recessed shape that engages with the subject P. Herein, the acoustic window 40 includes holding surfaces 41 to 44. The holding surface 41 engages with a surface either on the inside or the outside of a stretched second finger P2, for example, among the second to fifth fingers P2 to P5 of a long and thin long hand of the subject P. The holding surface 42 engages with a surface either on the inside or the outside of the third finger P3 that has been stretched so as to be approximately parallel to the second finger P2. The holding surface 43 engages with a surface either on the inside or the outside of the fourth finger P4 that has been stretched so as to be approximately parallel to the third finger P3. The holding surface 44 engages with a surface either on the inside or the outside of the fifth finger P5 that has been stretched so as to be approximately parallel to the fourth finger P4.

A recessed surface in a circular arc form laterally curving in accordance with the size of the second finger P2 is formed on the holding surface 41. A recessed surface in a circular arc form laterally curving in accordance with the size of the third finger P3 is formed on the holding surface 42. A recessed surface in a circular arc form laterally curving in accordance with the size of the fourth finger P4 is formed on the holding surface 43. And a recessed surface in a circular arc form laterally curving in accordance with the size of the fifth finger P5 is formed on the holding surface 44. The recessed surfaces formed respectively in the holding surfaces 41 to 44 are curved in a circular arc form with a radius ranging from 5 to 20 mm in accordance with the size of the fingers corresponding to the second to fifth fingers P2 to P5. Furthermore, the recessed surfaces respectively formed in the holding surfaces 41 to 44 are formed such that the longitudinal directions thereof are parallel to each other.

As described above, on a contacting surface of the acoustic window 40 that comes into contact with the second to fifth fingers P2 to P5 of the subject P, by forming holding the surfaces 41 to 44 in the recessed surface shape engaging with the second to fifth fingers P2 to P5, the gap between the second to fifth fingers P2 to P5 and the acoustic window 40 is reduced, making close contact possible.

Furthermore, by forming the holding surfaces 41 to 44 using a hard plastic material rather than using an elastic water bag, deformation in each of the holding surfaces 41 to 44 can be prevented and the durability can also be enhanced.

It should be noted that in the case of the holding surfaces 41 to 44 with which the inside surface of the second to fifth fingers P2 to P5 is caused to be in contact, it is possible to implement by forming a projected surface curving in each longitudinal direction of the holding surfaces 41 to 44. Whereby, since holding becomes possible in a natural state without stretching each of the second to fifth fingers P2 to P5, the load on the subject P can be reduced.

The transducer part 20 is arranged movably in the space closed by the acoustic window 40 and the casing 65, with transducers linearly arranged in one row in the longitudinal direction of the holding surfaces 41 to 44 of the acoustic window 40. Via an acoustic media that propagates ultrasound waves filled into the acoustic window 40 as well as the space, the transducer part 20 transceives ultrasound waves with respect to the second to fifth fingers P2 to P5 of the subject P while the inside or outside surface thereof is in contact with the holding surfaces 41 to 44.

The moving mechanism 60 includes a motor which is a power source to move the transducer part 20, a transmission mechanism, such as a gear, to transmit the power from the motor to the transducer part 20, a linear guide rail to guide the moving direction of the transducer part 20, and the like. Further, the moving mechanism 60 linearly moves the transducer part 20 between the holding surface 41 and the holding surface 44 in an arrow L1 direction as well as an arrow L2 direction opposite to the L1 direction. The arrow L1 direction is the direction vertical to the arrangement direction of the transducers and lateral to the holding surfaces 41 to 44.

It should be noted that in the case that the acoustic window 40 has one holding surface among the holding surfaces 41 to 44, by providing a transducer part having a plurality of transducers arranged in one row in a circular arc form in the lateral direction of the one holding surface, it can be implemented so as to cause the transducer part to move in the longitudinal direction of the holding surface which is the direction vertical to the arrangement direction of the transducers.

FIGS. 4A to 4C are drawings depicting other example of the configuration of an ultrasound probe. A plane drawing of an ultrasound probe 10a is depicted in FIG. 4A. Further, a cross-sectional drawing taken along arrows A-A of FIG. 4A is depicted in FIG. 4B, and a cross-sectional drawing taken along arrows B-B of FIG. 4A is depicted in FIG. 4C. In the ultrasound probe 10a, the same symbol is attached for units having the similar configuration and the similar function to each unit of the ultrasound probe 10 depicted in FIGS. 3A to 3C, and the detailed description thereof is omitted.

An aspect in the ultrasound probe 10a different from the ultrasound probe 10 is that the transducer part 20, the moving mechanism 60, and a casing 65 have been replaced with a transducer part 20a, a moving mechanism 60a, and the casing 65a. The transducer part 20a includes a plurality of transducers linearly arranged in one row in the lateral direction of the holding surfaces 41 to 44 of the acoustic window 40. The moving mechanism 60a causes the transducer part 20a to move linearly between one end and the other end in the longitudinal direction of the holding surfaces 41 to 44 in an arrow L3 direction as well as an arrow L4 direction opposite to the L3 direction. The arrow L3 direction is the direction vertical to the arrangement direction of the transducers. The casing 65a holds the moving mechanism 60a and the acoustic window 40.

Hereinafter, one example of the operation of the ultrasound diagnosis apparatus 100 is described with reference to FIGS. 1 to 5.

After conducting, from the operation part 98, an input of the imaging conditions of the subject P, an input of the ID of the subject P, and an input of the identification numbers of the holding surfaces 41 to 44 of the ultrasound probe 10 to be in contact with, for example, the inside surface of the second to fifth fingers P2 to P5 of the subject P which is an imaging object, the second to fifth fingers P2 to P5 are pressed against the holding surfaces 41 to 44 by applying jelly to the second to fifth fingers P2 to P5 or the holding surfaces 41 to 44. It should be noted that it is also possible to implement by causing the outside surface of the second to fifth fingers P2 to P5 to come into contact with the holding surfaces 41 to 44 depending on the imaging object.

As described above, the second to fifth fingers P2 to P5 can be in close contact with the acoustic window 40, thereby reducing the effort to apply jelly to the acoustic window 40 or the second to fifth fingers P2 to P5, and the effort to remove the jelly stuck on the acoustic window 40 or on the second to fifth fingers P2 to P5. Further, mixing in air that obstructs the propagation of ultrasound waves can be prevented from entering between the second to fifth fingers P2 to P5 and the acoustic window 40. Furthermore, unnecessary movement of the second to fifth fingers P2 to P5 can also be suppressed.

Once an input to start an examination is entered from the operation part 98, instructions for the examination are provided to each unit by the system controller 99. The moving mechanism 60 moves the transducer part 20 to a position corresponding to any one of the holding surfaces 41 to 44 identified by an input identification number.

The transceiver 80 processes reception signals obtained from the transducer part 20 at a plurality of positions with regard to each of the holding surfaces 41 to 44. The signal processor 90 generates data for an imaging region. The image data generator 91 generates three-dimensional image data corresponding to each of the holding surfaces 41 to 44 by scanning conversion of the data of each imaging region. The display 92 displays all image data generated by the image data generator 91.

FIG. 5 is a drawing depicting one example of a screen displaying data for each image on the display 92. A screen 93 consists of first to fourth display areas 931 to 934 respectively related to holding surfaces 41 to 44. “1” which is the identification number of the holding surface 41 and image data 94 of the second finger P2 held in contact with the holding surface 41 are displayed in the first display area 931. “2” which is the identification number of the holding surface 42 and image data 95 of the third finger P3 held in contact with the holding surface 42 are displayed in the second display area 932. “3” which is the identification number of the holding surface 43 and image data 96 of the fourth finger P4 held in contact with the holding surface 43 are displayed in the third display area 933. Also, “4” which is the identification number of the holding surface 44 and image data 97 of the fifth finger P5 held in contact with the holding surface 44 are displayed in the fourth display area 934.

It should be noted that FIG. 5 was described presuming that the image data 94 to 97 is displayed in the first to fourth display areas 931 to 934 of the display 92; however, in place of the image data 94 to 97, or in addition to the image data 94 to 97, it is also possible to display images, and the like, obtained by Doppler waves based on reflected waves from the second to fifth fingers P2 to P5 or by CFM (Color Flow Mapping) process.

As described above, by linking the holding surfaces 41 to 44 and the first to the fourth display areas 931 to 934 of the display 92, it is possible to easily identify that the image data 94 to 97 displayed in the first to fourth display areas 931 to 934 of the display 92 is data of the second to fifth fingers P2 to P5.

Further, because mixing in air that obstructs the propagation of ultrasound waves can be prevented from entering between the second to fifth fingers P2 to P5 and the acoustic window 40, deteriorating image quality of the image data 94 to 97 of the second to fifth fingers P2 to P5 can be prevented. Furthermore, unnecessary movement of the second to fifth fingers P2 to P5 can also be suppressed, enabling the prevention of the image quality of each image data 94 to 97 from deteriorating due to an artifact.

According to the first embodiment described above, on the contacting surface of the acoustic window 40 that comes into contact with the second to fifth fingers P2 to P5, by forming the holding surfaces 41 to 44 in a recessed surface shape engaging the second to fifth fingers P2 to P5, close contact between the second to fifth fingers P2 to P5 and the acoustic window 40 becomes possible.

Thereby, because the amount of jelly to be interposed between the second to fifth fingers P2 to P5 and the acoustic window 40 can be reduced, the effort to apply jelly to the acoustic window 40 or the second to fifth fingers P2 to P5 and the effort to remove the jelly stuck on the acoustic window 40 or the second to fifth fingers P2 to P5 can be reduced. Further, because mixing in air that obstructs the propagation of ultrasound waves can be prevented from entering between the second to fifth fingers P2 to P5 and the acoustic window 40, deteriorating image quality of the image data of the second to fifth fingers P2 to P5 can be prevented. Furthermore, unnecessary movement of the second to fifth fingers P2 to P5 can also be suppressed, enabling the prevention of the image quality of the image data from deteriorating due to the artifact.

### Second Embodiment

FIGS. 6A to 6C are drawings depicting the configuration of an ultrasound probe of the ultrasound diagnosis apparatus according to a second embodiment. A plane drawing of an ultrasound probe 10b is depicted in FIG. 6A, a cross-sectional drawing taken along arrows A-A of FIG. 6A is depicted in FIG. 6B, and a cross-sectional drawing taken along arrows B-B of FIG. 6A is depicted in FIG. 6C. In the ultrasound probe 10b, the same symbol is attached for units having the similar configuration and the similar function to each unit of the ultrasound probe 10 of the first embodiment depicted in FIGS. 3A to 3C, and the detailed description thereof is omitted.

The ultrasound probe 10b includes an acoustic window 50 that comes into contact with the subject P, the transducer part 20, a moving mechanism 60b for moving the transducer part 20, and a casing 65b for holding moving the mechanism 60b as well as the acoustic window 50.

The acoustic window 50 is made from the same material as the acoustic window 40 of the ultrasound probe 10, and has more than one holding surface in a recessed shape that engages with the subject P. Herein, the acoustic window 50 has holding surfaces 51 to 54. The holding surface 51 engages with a surface either on the inside or the outside of the spread and stretched second finger P2 among the second to fifth fingers P2 to P5 of the subject P. The holding surface 52 engages with a surface either on the inside or the outside of the third finger P3. The holding surface 53 engages with a surface either on the inside or the outside of the fourth finger P4. Also, the holding surface 54 engages with a surface either on the inside or the outside of the fifth finger P5.

A recessed surface in a circular arc form laterally curving in accordance with the size of the second finger P2 is formed on the holding surface 51. A recessed surface in a circular arc form laterally curving in accordance with the size of the third finger P3 is formed on the holding surface 52. A recessed surface in a circular arc form laterally curving in accordance with the size of the fourth finger P4 is formed on the holding surface 53. Also, a recessed surface in a circular arc form laterally curving in accordance with the size of the fifth finger P5 is formed on the holding surface 54. The recessed surfaces formed respectively in the holding surfaces 51 to 54 are curved in a circular arc form with a radius ranging from 5 to 20 mm in accordance with the size of the finger corresponding to the second to fifth fingers P2 to P5. Furthermore, the recessed surfaces respectively formed in the holding surfaces 51 to 54 are formed such that the longitudinal directions thereof are radial to each other corresponding to the spread and stretched second to fifth fingers P2 to P5.

As described above, on a contacting surface of the acoustic window 50 that comes into contact with the second to fifth fingers P2 to P5 of the subject P, by forming the holding surfaces 51 to 54 in the recessed surface shape engaging with the second to fifth fingers P2 to P5, the gap between the second to fifth fingers P2 to P5 and the acoustic window 50 is reduced, making close contact possible.

Furthermore, by forming the holding surfaces 51 to 54 using a hard plastic material rather than using an elastic water bag, deformation in each of the holding surfaces 51 to 54 can be prevented and the durability can also be enhanced.

The transducer part 20 is arranged such that it is movable in the space closed by the acoustic window 50 and the casing 65b, and transceiver, via the acoustic window 50 and an acoustic medium filled into the space, ultrasound waves with respect to the second to fifth fingers P2 to P5 while the inside or outside surface thereof is in contact with the holding surfaces 51 to 54.

The moving mechanism 60b is constituted as same as the moving mechanism 60 of the ultrasound probe 10. The moving mechanism 60b linearly moves the transducer part 20 between the holding surface 51 and the holding surface 54 in the arrow L1 direction as well as the arrow L2 direction. The arrow L1 direction is the direction vertical to the arrangement direction of the transducers including directions other than the direction vertical to the longitudinal direction of the holding surfaces 51 to 54 of the acoustic window 50.

According to the second embodiment described above, on the contacting surface of the acoustic window 50 that comes into contact with the second to fifth fingers P2 to P5, by forming the holding surfaces 51 to 54 in a recessed surface shape engaging with the second to fifth fingers P2 to P5, close contact between the second to fifth fingers P2 to P5 and the acoustic window 50 becomes possible.

Thereby, because the amount of jelly to be interposed between the second to fifth fingers P2 to P5 and the acoustic window 50 can be reduced, the effort to apply jelly to the acoustic window 50 or the second to fifth fingers P2 to P5 and the effort to remove the jelly stuck on the acoustic window 50 or the second to fifth fingers P2 to P5 can be reduced. Further, because mixing in air that obstructs the propagation of ultrasound waves can be prevented from entering between the second to fifth fingers P2 to P5 and the acoustic window 50, deteriorating image quality of the image data of the second to fifth fingers P2 to P5 can be prevented. Furthermore, unnecessary movement of the second to fifth fingers P2 to P5 may also be suppressed, enabling the prevention of the image quality of the image data from deteriorating due to an artifact.

### Third Embodiment

FIGS. 7A to 7C and FIG. 8 are drawings depicting the configuration of an ultrasound probe of the ultrasound diagnosis apparatus according to a third embodiment. A plane drawing of an ultrasound probe 10c is depicted in FIG. 7A, a cross-sectional drawing taken along arrows A-A of FIG. 7A is depicted in FIG. 7B, and a cross-sectional drawing taken along arrows B-B of FIG. 7A is depicted in FIG. 7C. Further, FIG. 8 shows a cross-sectional drawing taken along arrows C-C of FIG. 7C. In the ultrasound probe 10c, the same symbol is attached for units having the similar configuration and the similar function to each unit of the ultrasound probe 10 of the first embodiment depicted in FIGS. 3A to 3C and the ultrasound probe 10b of the second embodiment depicted in FIGS. 6A to 6C, and the detailed description thereof is omitted.

The ultrasound probe 10c includes the acoustic window 50, the transducer part 20, a moving mechanism 60c for the moving transducer part 20, and a casing 65c for holding the moving mechanism 60c as well as the acoustic window 50.

The transducer part 20 is arranged movably in the space closed by the acoustic window 50 and the casing 65c, and transceives, via the acoustic window 50 and an acoustic medium filled into the space, ultrasound waves with respect to the second to fifth fingers P2 to P5 while the inside or outside surface thereof is in contact with the holding surfaces 51 to 54.

The moving mechanism 60c includes a motor which is a power source to move the transducer part 20, a transmission mechanism, such as a gear, to transmit the power from the motor to the transducer part 20, a guide rail in a circular arc form to guide the moving direction of the transducer part 20, and the like. The moving mechanism 60c moves the transducer part 20 in a circular arc form in an arrow R1 direction and an arrow R2 direction opposite to the R1 direction. The arrow R1 direction is the direction vertical to the longitudinal direction of the holding surface at each position of the holding surfaces 51 to 54, and is the direction vertical to the arrangement direction of the transducers.

According to the third embodiment described above, on the contacting surface of the acoustic window 50 that comes into contact with the second to fifth fingers P2 to P5, by forming the holding surfaces 51 to 54 in a recessed surface shape engaging the second to fifth fingers P2 to P5, close contact between the second to fifth fingers P2 to P5 and the acoustic window 40 becomes possible.

Thereby, because the amount of jelly to be interposed between the second to fifth fingers P2 to P5 and the acoustic window 50 can be reduced, the effort to apply jelly to the acoustic window 50 or the second to fifth fingers P2 to P5 and the effort to remove the jelly stuck on the acoustic window 50 or the second to fifth fingers P2 to P5 can be reduced. Further, because mixing in air that obstructs the propagation of ultrasound waves can be prevented from entering between the second to fifth fingers P2 to P5 and the acoustic window 50, deteriorating image quality of the image data of the second to fifth fingers P2 to P5 can be prevented. Furthermore, unnecessary movement of the second to fifth fingers P2 to P5 can also be suppressed, enabling the prevention of the image quality of the image data from deteriorating due to an artifact.

### Fourth Embodiment

FIG. 9 and FIGS. 10A to 10B are drawings depicting the configuration of an ultrasound probe of the ultrasound diagnosis apparatus according to a fourth embodiment. A plane drawing of an ultrasound probe 10d is depicted in FIG. 9. Further, a cross-sectional drawing taken along arrows A-A of FIG. 9 is depicted in FIG. 10A, and a cross-sectional drawing taken along arrows B-B of FIG. 9 is depicted in FIG. 10B. In the ultrasound probe 10d, the same symbol is attached for units having the similar configuration and the similar function to each unit of the ultrasound probe 10b of the second embodiment depicted in FIGS. 6A to 6C, and the detailed description thereof is omitted.

The ultrasound probe 10d includes the acoustic window 50, the same number of first to fourth transducer parts 31 to 34 as the holding surfaces 51 to 54 corresponding to each of the holding surfaces 51 to 54 of the acoustic window 50, a moving mechanism 60d for moving each of the first to fourth transducer parts 31 to 34, and a casing 65d for holding the moving mechanism 60d as well as the acoustic window 50.

Each of the first to fourth transducer parts 31 to 34 is arranged movably in the space closed by the acoustic window 50 and the casing 65d. The first transducer part 31 is arranged close to the holding surface 51, and has a plurality of transducers linearly arranged in one row in the longitudinal direction of the holding surface 51. Moreover, the first transducer part 31 transceives, via the acoustic window 50 and an acoustic medium, ultrasound waves with respect to the second finger P2 while the inside or outside surface thereof is in contact with the holding surface 51. The second transducer part 32 is arranged close to the holding surface 52, and has a plurality of transducers linearly arranged in one row in the longitudinal direction of the holding surface 52. Moreover, the second transducer part 32 transceives, via the acoustic window 50 and an acoustic medium, ultrasound waves with respect to the third finger P3 while the inside or outside surface thereof is in contact with the holding surface 52.

The third transducer part 33 is arranged close to the holding surface 53, and has a plurality of transducers linearly arranged in one row in the longitudinal direction of the holding surface 53. Moreover, the third transducer part 33 transceives, via the acoustic window 50 and an acoustic medium, ultrasound waves with respect to the fourth finger P4 while the inside or outside surface thereof is in contact with the holding surface 53. Also, the fourth transducer part 34 is arranged close to the holding surface 54, and has a plurality of transducers linearly arranged in one row in the longitudinal direction of the holding surface 54. Moreover, the fourth transducer part 34 transceives, via the acoustic window 50 and an acoustic medium, ultrasound waves with respect to the fifth finger P5 while the inside or outside surface thereof is in contact with the holding surface 54.

The moving mechanism 60d includes a motor which as a power source to move each of the first to fourth transducer parts 31 to 34, a transmission mechanism, such as a gear, to transmit the power from the motor to the first to fourth transducer parts 31 to 34, and the like. As shown in FIG. 10A, the moving mechanism 60d moves the first transducer part 31 in a circular arc form in an arrow R3 direction which is the direction vertical to the arrangement direction of the transducers along the holding surface 51 and an arrow R4 direction opposite to the R3 direction.

Furthermore, the moving mechanism 60d moves the second transducer part 32 along the holding surface 52 in a circular arc form in an arrow R5 direction which is the direction vertical to the arrangement direction of the transducers and an arrow R6 direction opposite to the R5 direction. The moving mechanism 60d also moves the third transducer part 33 along the holding surface 53 in a circular arc form in an arrow R7 direction which is the direction vertical to the arrangement direction of the transducers and an arrow R8 direction opposite to the R7 direction. Moreover, the moving mechanism 60d moves the fourth transducer part 34 along the holding surface 54 in a circular arc form in an arrow R9 direction which is the direction vertical to the arrangement direction of the transducers and an arrow R10 direction opposite to the R9 direction.

According to the fourth embodiment described above, on the contacting surface of the acoustic window 50 that comes into contact with the second to fifth fingers P2 to P5, by forming the holding surfaces 51 to 54 in a recessed surface shape engaging the second to fifth fingers P2 to P5, close contact between the second to fifth fingers P2 to P5 and the acoustic window 50 becomes possible.

Thereby, because the amount of jelly to be interposed between the second to fifth fingers P2 to P5 and the acoustic window 50 may be reduced, the effort to apply jelly to the acoustic window 50 or the second to fifth fingers P2 to P5 and the effort to remove the jelly stuck on the acoustic window 50 or the second to fifth fingers P2 to P5 can be reduced. Further, because mixing in air that obstructs the propagation of ultrasound waves can be prevented from entering between the second to fifth fingers P2 to P5 and the acoustic window 50, deteriorating image quality of the image data of the second to fifth fingers P2 to P5 can be prevented. Furthermore, unnecessary movement of the second to fifth fingers P2 to P5 can also be suppressed, enabling the prevention of the image quality of the image data from deteriorating due to an artifact.

### Fifth Embodiment

FIGS. 11A to 11C are drawings depicting the configuration of an ultrasound probe of the ultrasound diagnosis apparatus according to a fifth embodiment. A plane drawing of an ultrasound probe 10e is depicted in FIG. 11A, a cross-sectional drawing taken along arrows A-A of FIG. 11A is depicted in FIG. 11B, and a cross-sectional drawing taken along arrows B-B of FIG. 11A is depicted in FIG. 11C. In the ultrasound probe 10e, the same symbol is attached for units having the similar configuration and the similar function to each unit of the ultrasound probe 10b of the second embodiment depicted in FIGS. 6A to 6C, and the detailed description thereof is omitted.

The ultrasound probe 10e includes the acoustic window 50, the same number of first to fourth transducer parts 31a to 34a as the holding surfaces 51 to 54 corresponding to each of the holding surfaces 51 to 54 of the acoustic window 50, a moving mechanism 60e for moving the first to fourth transducer parts 31a to 34a, and a casing 65e for holding the moving mechanism 60e as well as the acoustic window 50.

Each of the first to fourth transducer parts 31a to 34a is arranged movably in the space closed by the acoustic window 50 and the casing 65e. The first transducer part 31a is arranged close to the holding surface 51 and has a plurality of transducers arranged in a circular arc form in one row in the lateral direction of the holding surface 51. Moreover, the first transducer part 31a transceives, via the acoustic window 50 and an acoustic medium, ultrasound waves with respect to the second finger P2 while the inside or outside surface thereof is in contact with the holding surface 51. The second transducer part 32a is arranged close to the holding surface 52, and has a plurality of transducers arranged in a circular arc form in one row in the lateral direction of the holding surface 52. Moreover, the second transducer part 32a transceives, via the acoustic window 50 and an acoustic medium, ultrasound waves with respect to the third finger P3 while the inside or outside surface thereof is in contact with the holding surface 52.

The third transducer part 33a is arranged close to the holding surface 53, and has a plurality of transducers arranged in a circular arc form in one row in the lateral direction of the holding surface 53. Moreover, the third transducer part 33a transceives, via the acoustic window 50 and an acoustic medium, ultrasound waves with respect to the fourth finger P4 while the inside or outside surface thereof is in contact with the holding surface 53. Also, the fourth transducer part 34a is arranged close to the holding surface 54, and has a plurality of transducers arranged in a circular arc form in one row in the lateral direction of the holding surface 54. Moreover, the fourth transducer part 34a transceives, via the acoustic window 50 and an acoustic medium, ultrasound waves with respect to the fifth finger P5 while the inside or outside surface thereof is in contact with the holding surface 54.

The moving mechanism 60e includes a motor which is a power source to move each of the first to fourth transducer parts 31a to 34a, a transmission mechanism, such as a gear, to transmit the power from the motor to the first to fourth transducer parts 31a to 34a, and the like. The moving mechanism 60e linearly moves the first transducer part 31a in the longitudinal direction of the holding surface 51 that is vertical to the arrangement direction of the transducers. The moving mechanism 60e linearly moves the second transducer part 32a in an arrow L5 direction which is the longitudinal direction of the holding surface 52 which is vertical to the arrangement direction of the transducers and an arrow L6 direction opposite to the L5 direction. The moving mechanism 60e linearly moves the third transducer part 33a in the longitudinal direction of the holding surface 53 which is vertical to the arrangement direction of the transducers. The moving mechanism 60e also linearly moves the fourth transducer part 34a in the longitudinal direction of the holding surface 54 which is vertical to the arrangement direction of the transducers.

According to the fifth embodiment described above, on the contacting surface of the acoustic window 50 that comes into contact with the second to fifth fingers P2 to P5, by forming the holding surfaces 51 to 54 in a recessed surface shape engaging the second to fifth fingers P2 to P5, close contact between the second to fifth fingers P2 to P5 and the acoustic window 50 becomes possible.

Thereby, because the amount of jelly to be interposed between the second to fifth fingers P2 to P5 and the acoustic window 50 can be reduced, the effort to apply jelly to the acoustic window 50 or the second to fifth fingers P2 to P5 and the effort to remove the jelly stuck on the acoustic window 50 or the second to fifth fingers P2 to P5 can be reduced. Further, because mixing in air that obstructs the propagation of ultrasound waves can be prevented from entering between the second to fifth fingers P2 to P5 and the acoustic window 50, deteriorating image quality of the image data of the second to fifth fingers P2 to P5 can be prevented. Furthermore, unnecessary movement of the second to fifth fingers P2 to P5 can also be suppressed, enabling the prevention of the image quality of the image data from deteriorating due to an artifact.

### Sixth Embodiment

FIGS. 12A to 12C are drawings depicting the configuration of an ultrasound probe of the ultrasound diagnosis apparatus according to a sixth embodiment. A plane drawing of an ultrasound probe 10f is depicted in FIG. 12A, a cross-sectional drawing taken along arrows A-A of FIG. 12A is depicted in FIG. 12B, and a cross-sectional drawing taken along arrows B-B of FIG. 12A is depicted in FIG. 12C. In the ultrasound probe 10f, the same symbol is attached for units having the similar configuration and the similar function to each unit of the ultrasound probe 10 of the first embodiment depicted in FIGS. 3A to 3C, and the detailed description thereof is omitted.

The ultrasound probe 10f includes a first ultrasound probe 11 for transceiving ultrasound waves by contacting a surface on one side either inside or outside of the second to fifth fingers P2 to P5 of the subject P, and a second ultrasound probe 12 for transceiving ultrasound waves by contacting the surface on the other side of the second to fifth fingers P2 to P5.

The first ultrasound probe 11 includes the acoustic window 40 engaging the inside or outside surface of the second to fifth fingers P2 to P5, the transducer part 20, the moving mechanism 60, a casing 65f1 for holding the moving mechanism 60 as well as the acoustic window 40, and a rotary shaft 66 supported by the casing 65f1. That is, the first ultrasound probe 11 may include first acoustic window having a first holding surface in a recessed surface shape and a first transducer part having a plurality of first transducers for transceiving, via the first acoustic window, ultrasound waves with respect to a subject that comes into contact with the first holding surface.

The second ultrasound probe 12 is, with respect to first ultrasound probe 11, rotatably supported by the rotary shaft 66 in an arrow R11 direction and an arrow R12 opposite to the R11 direction. Further, the second ultrasound probe 12 includes the acoustic window 40 engaging the inside or outside surface of the second to fifth fingers P2 to P5 arranged so that the second to fifth fingers P2 to P5 can be covered with the acoustic window 40 of the first ultrasound probe 11, the transducer part 20, and the moving mechanism 60, which are symmetrical to the acoustic window 40, the transducer part 20, and the moving mechanism 60 of the first ultrasound probe 11, based on the contacting surface with the first ultrasound probe 11 when rotated in the R11 direction to an angle (closing angle) contacting the first ultrasound probe 11. Furthermore, the second ultrasound probe 12 includes the moving mechanism 60 and a casing 65f2 for holding the acoustic window 40. That is, the second ultrasound probe 12 may include a second acoustic window having a second holding surface in a recessed surface shape and a second transducer part having a plurality of second transducers for transceiving, via the second acoustic window, ultrasound waves with respect to a subject that comes into contact with the second holding surface.

Moreover, in the case that the lesion of the subject P is not clear, for example, whether inside or outside of the second to fifth fingers P2 to P5 and image data for both sides is required, by rotating the second ultrasound probe 12 in the R11 direction to the closing angle while, for example, the inside surface of the second to fifth fingers P2 to P5 is in a state of contacting the holding surfaces 41 to 44 of the acoustic window 40 of the first ultrasound probe 11, the ultrasound probe 10f holds the second to fifth fingers P2 to P5 by causing the holding surfaces 41 to 44 of the acoustic window 40 of the second ultrasound probe 12 to contact the outside surface thereof.

The ultrasound probe 10f then generates first image data by transceiving ultrasound waves by moving one of the transceiving parts 20 of the first and second ultrasound probes 11, 12 while each of the acoustic windows 40 of the first and second ultrasound probes 11, 12 is in a state of contacting the second to fifth fingers P2 to P5 by surrounding from inside and outside, and generates second image data by moving the other transducer part 20 after moving of the former transducer part 20 to transceive ultrasound waves. Subsequently, the ultrasound probe 10f generates image data capable of observing both the inside and outside of the second to fifth fingers P2 to P5 by synthesizing the first image data and the second image data.

In the case that the first and second image data on the inside as well as the outside of the second to fifth fingers P2 to P5 is required, because it is possible to transceive ultrasound waves from both sides of the second to fifth fingers P2 to P5 while the second to fifth fingers P2 to P5 are in a state of contacting the holding surfaces 41 to 44, the work hours can be cut short.

It should be noted that implementation may also be possible by a ultrasound probe consisting of a first ultrasound probe including, shown in FIGS. 6A to 6C, the acoustic window 50, the transducer part 20, the moving mechanism 60b and a casing for holding the moving mechanism 60b as well as the acoustic window 50, and a second ultrasound probe including the acoustic window 50, the transducer part 20, the moving mechanism 60b, and a casing for holding the moving mechanism 60b and the acoustic window 50 which are arranged symmetrically with respect to the acoustic window 50, the transducer part 20, and the moving mechanism 60b of the first ultrasound probe, based on the contacting surface to contact the first ultrasound probe.

According to the sixth embodiment described above, on the contacting surface of the acoustic window 40 that comes into contact with the second to fifth fingers P2 to P5, by forming the holding surfaces 41 to 44 in a recessed surface shape engaging the second to fifth fingers P2 to P5, close contact between the second to fifth fingers P2 to P5 and the acoustic window 40 becomes possible.

Thereby, because the amount of jelly to be interposed between the second to fifth fingers P2 to P5 and the acoustic window 40 can be reduced, the effort to apply jelly to the acoustic window 40 or the second to fifth fingers P2 to P5 and the effort to remove the jelly stuck on the acoustic window 40 or the second to fifth fingers P2 to P5 can be reduced. Further, because mixing in air that obstructs the propagation of ultrasound waves can be prevented from entering between the second to fifth fingers P2 to P5 and the acoustic window 40, deteriorating image quality of the image data of the second to fifth fingers P2 to P5 can be prevented. Furthermore, unnecessary movement of the second to fifth fingers P2 to P5 can also be suppressed, enabling the prevention of the image quality of the image data from deteriorating due to an artifact.

Also, in the case that the second to fifth fingers P2 to P5 are imaging objects when the inside as well as the outside surface of the fingers are in contact with the holding surfaces 41 to 44, by providing the first and second ultrasound probes 11, 12, ultrasound waves can be transceived from both sides of the second to fifth fingers P2 to P5, making it possible to cut the work hours short.

### Seventh Embodiment

FIGS. 13A to 13C are drawings depicting the configuration of an ultrasound probe of the ultrasound diagnosis apparatus according to a seventh embodiment. A plane drawing of an ultrasound probe 10g is depicted in FIG. 13A, a cross-sectional drawing taken along arrows A-A of FIG. 13A is depicted in FIG. 13B, and a cross-sectional drawing taken along arrows B-B of FIG. 13A is depicted in FIG. 13C. In the ultrasound probe 10g, the same symbol is attached for units having the similar configuration and the similar function to each unit of the ultrasound probe 10 of the first embodiment depicted in FIGS. 3A to 3C, and the detailed description thereof is omitted.

The ultrasound probe 10g includes the acoustic window 40, a transducer part 20g having a plurality of transducers, and a casing 65g for holding the acoustic window 40 and the transducer part 20g.

The transducer part 20g is arranged in the space closed by the acoustic window 40 and the casing 65g, while the transducers are arranged in a two-dimensional array form in both the longitudinal and lateral directions of the holding surfaces 41 to 44 of the acoustic window 40. Specifically, a group of transducers in each row linearly arranged in the longitudinal direction of the holding surfaces 41 to 44 is arranged by forming more than two rows in the lateral direction. Moreover, the transducer part 20g transceives, via the acoustic window 40 and an acoustic medium filled into the space, ultrasound waves with respect to the second to fifth fingers P2 to P5 while the inside or outside surface thereof is in contact with the holding surfaces 41 to 44.

A plurality of transducers included in the transducer part 20g simply has to be arranged such that the transducers are capable of transceiving ultrasound waves with respect to the fingers contacting each of the holding surfaces 41 to 44 of the acoustic window 40.

For example, a plurality of transducers is arranged in a two-dimensional array form such that the transducers overlap the holding surfaces 41 to 44 of the acoustic window 40 when viewed from the top surface. In this case, among the arranged plurality of transducers, those transducers that transceive ultrasound waves with respect to the second to fifth fingers P2 to P5 contacting the holding surfaces 41 to 44 are subjected to electronic scanning control by the transceiver 80.

Furthermore, for example, a plurality of transducers is arranged in a two-dimensional array form almost over the entire lower surface of each of the holding surfaces 41 to 44 of the acoustic window 40. In this case, among the plurality of the transducers arranged over the entire surface, only those transducers that transceive ultrasound waves with respect to the second to fifth fingers P2 to P5 contacting the holding surfaces 41 to 44 are subjected to electronic scanning control by the transceiver 80.

To the transducer part 20g, the transmitter 81 generates a rate pulse for each channel to determine the repetition cycle of ultrasound waves to radiate the subject P. To each generated rate pulse, the transmitter 81 provides a delay time, for example, to be determined for each transducer according to the transmitting direction and the transmission focus position. Moreover, the transmitter 81 generates a drive pulse at a timing based on each delayed rate pulse, and supplies the generated drive pulse to each transducer. Each transducer to which the drive pulse has been supplied generates ultrasound waves.

The receiver 82 secures a sufficient S/N by amplifying minute reception signals obtained from the transducer part 20g. The receiver 82 then provides, to the amplified reception signals, a delay time for converging reflected waves from a predetermined depth and a delay time to set the reception directionality by sequentially making changes, performs phasing and addition on the reception signals to generate one signal, and outputs the signal to the signal processor 90.

It should be noted that the holding surfaces 41 to 44 of the acoustic windows 40 may also be the holding surfaces 51 to 54 of the acoustic window 50 formed such that the longitudinal directions thereof are mutually in a radial form as depicted in FIGS. 6A to 6C.

According to the seventh embodiment described above, on the contacting surface of the acoustic window 40 that comes into contact with the second to fifth fingers P2 to P5, by forming the holding surfaces 41 to 44 in a recessed surface shape engaging the second to fifth fingers P2 to P5, close contact between the second to fifth fingers P2 to P5 and the acoustic window 40 becomes possible.

Thereby, because the amount of jelly to be interposed between the second to fifth fingers P2 to P5 and the acoustic window 40 can be reduced, the effort to apply jelly to the acoustic window 40 or the second to fifth fingers P2 to P5 and the effort to remove the jelly stuck on the acoustic window 40 or the second to fifth fingers P2 to P5 can be reduced. Further, because mixing in air that obstructs the propagation of ultrasound waves can be prevented from entering between the second to fifth fingers P2 to P5 and the acoustic window 40, deteriorating image quality of the image data of the second to fifth fingers P2 to P5 can be prevented. Furthermore, unnecessary movement of the second to fifth fingers P2 to P5 can also be suppressed, enabling the prevention of the image quality of the image data from deteriorating due to an artifact. Moreover, since the transducer part does not have to be moved by the moving mechanism, the space filled in with an acoustic medium can be made small, and enabling the further prevention of deteriorating image quality of the image data of the second to fifth fingers P2 to P5, and reduction in the size as well as the cost of the ultrasound probe 10g.

### Eighth Embodiment

FIGS. 14A to 14C are drawings depicting the configuration of an ultrasound probe of the ultrasound diagnosis apparatus according to an eighth embodiment. A plane drawing of an ultrasound probe 10h is depicted in FIG. 14A, a cross-sectional drawing taken along arrows A-A of FIG. 14A is depicted in FIG. 14B, and a cross-sectional drawing taken along arrows B-B of FIG. 14A is depicted in FIG. 14C. In the ultrasound probe 10h, the same symbol is attached for units having the similar configuration and the similar function to each unit of the ultrasound probe 10e of the fifth embodiment depicted in FIGS. 11A to 11C, and the detailed description thereof is omitted.

The ultrasound probe 10h includes the acoustic window 50, the same number of first to fourth transducer parts 31h to 34h as the holding surfaces 51 to 54 corresponding to each of the holding surfaces 51 to 54 of the acoustic window 50, and a casing 65h for the holding acoustic window 50.

Each of the first to fourth transducer parts 31h to 34h is arranged movably in the space closed by the acoustic window 50 and the casing 65h. The first transducer part 31h is arranged close to the holding surface 51, and has a plurality of transducers arranged in a two-dimensional array form in both the longitudinal and lateral directions of the holding surface 51. Specifically, a group of transducers in each row linearly arranged in the longitudinal direction of the holding surface 51 is arranged by forming more than two rows in a circular arc form in the lateral direction. Moreover, the first transducer part 31h transceives, via the acoustic window 50 and an acoustic medium, ultrasound waves with respect to the second finger P2 while the inside or outside surface thereof is in contact with the holding surface 51. Further, the second transducer part 32h is arranged close to the holding surface 52, and has a plurality of transducers arranged in a two-dimensional array form in both the longitudinal and lateral directions of the holding surface 52. Specifically, a group of transducers in each row linearly arranged in the longitudinal direction of the holding surface 52 is arranged by forming more than two rows in a circular arc form in the lateral direction. Moreover, the second transducer part 32h transceives, via the acoustic window 50 and an acoustic medium, ultrasound waves with respect to the third finger P3 while the inside or outside surface thereof is in contact with the holding surface 52.

Further, the third transducer part 33h is arranged close to the holding surface 53, and has a plurality of transducers arranged in a two-dimensional array form in both the longitudinal and lateral directions of the holding surface 53. Specifically, a group of transducers in each row linearly arranged in the longitudinal direction of the holding surface 53 is arranged by forming more than two rows in a circular arc form in the lateral direction. Moreover, the third transducer part 33h transceives, via the acoustic window 50 and an acoustic medium, ultrasound waves with respect to the fourth finger P4 while the inside or outside surface thereof is in contact with the holding surface 53. Furthermore, the fourth transducer part 34h is arranged close to the holding surface 54, and has a plurality of transducers arranged in a two-dimensional array form in both longitudinal and lateral directions of the holding surface 54. Specifically, a group of transducers in each row linearly arranged in the longitudinal direction of the holding surface 54 is arranged by forming more than two rows in a circular arc form in the lateral direction. Moreover, the fourth transducer part 34h transceives, via the acoustic window 50 and an acoustic medium, ultrasound waves with respect to the fifth finger P5 while the inside or outside surface thereof is in contact with the holding surface 54.

As in the seventh embodiment, the first to fourth transducer parts 31h to 34h are subjected to electronic scanning control by the transmitter 80.

According to the eighth embodiment described above, on the contacting surface of the acoustic window 50 that comes into contact with the second to fifth fingers P2 to P5, by forming the holding surfaces 51 to 54 in a recessed surface shape engaging the second to fifth fingers P2 to P5, close contact between the second to fifth fingers P2 to P5 and the acoustic window 50 becomes possible.

Thereby, because the amount of jelly to be interposed between the second to fifth fingers P2 to P5 and the acoustic window 50 can be reduced, the effort to apply jelly to the acoustic window 50 or the second to fifth fingers P2 to P5 and the effort to remove the jelly stuck on the acoustic window 50 or the second to fifth fingers P2 to P5 can be reduced. Further, because mixing in air that obstructs the propagation of ultrasound waves can be prevented from entering between the second to fifth fingers P2 to P5 and the acoustic window 50, deteriorating image quality of the image data of the second to fifth fingers P2 to P5 can be prevented. Furthermore, unnecessary movement of the second to fifth fingers P2 to P5 can also be suppressed, enabling the prevention of the image quality of the image data from deteriorating due to an artifact. Moreover, since the transducer part does not have to be moved by the moving mechanism, the space filled in with an acoustic medium can be made small, and enabling further prevention of deteriorating image quality of the image data of the second to fifth fingers P2 to P5 and reduction in the size as well as the cost of the ultrasound probe 10g.

### Ninth Embodiment

FIGS. 15A to 15C are drawings depicting the configuration of an ultrasound probe of the ultrasound diagnosis apparatus according to a ninth embodiment. A plane drawing of an ultrasound probe 10j is depicted in FIG. 15A, a cross-sectional drawing taken along arrows A-A of FIG. 15A is depicted in FIG. 15B, and a cross-sectional drawing taken along arrows B-B of FIG. 15A is depicted in FIG. 15C. In the ultrasound probe 10j, the same symbol is attached for units having the similar configuration and the similar function to each unit of the ultrasound probe 10 of the first embodiment depicted in FIGS. 3A to 3C, and the detailed description thereof is omitted.

The ultrasound probe 10j includes the acoustic window 40 that comes into contact with the subject P, a transducer part 20j, a moving mechanism 60j for moving the transducer part 20j, and a casing 65j for holding the moving mechanism 60j as well as the acoustic window 40.

The acoustic window 40 includes more than one holding surface in a recessed surface shape that engages with the subject P. Herein, the acoustic window 40 has the holding surfaces 41 to 44.

The transducer part 20j is arranged movably in the space closed by the acoustic window 40 and the casing 65j, and the transducers are arranged in a two-dimensional array form in both the longitudinal and lateral directions of the holding surfaces 41 to 44 of the acoustic window 40. Specifically, a group of transducers in each row linearly arranged in the longitudinal direction of the holding surfaces 41 to 44 is arranged by forming more than two rows in the lateral direction. Moreover, the transducer part 20j transceives, via the acoustic window 40 and an acoustic medium filled into the space, ultrasound waves with respect to the second to fifth fingers P2 to P5 while the inside or outside surface thereof is in contact with the holding surfaces 41 to 44.

As in the seventh embodiment, the transducer part 20j is subjected to electronic scanning control by the transceiver 80.

The moving mechanism 60j is constituted as same as the moving mechanism 60 of the ultrasound probe 10, and moves the transducer part 20j in the direction vertical to the arrangement direction (longitudinal direction of the holding surface in the ninth embodiment) of each row of the transducers. Specifically, the moving mechanism 60j linearly moves the transducer part 20j between the holding surface 41 and the holding surface 44 in the L1 direction as well as the L2 direction.

According to the ninth embodiment described above, on the contacting surface of the acoustic window 40 that comes into contact with the second to fifth fingers P2 to P5, by forming the holding surfaces 41 to 44 in a recessed surface shape engaging the second to fifth fingers P2 to P5, close contact between the second to fifth fingers P2 to P5 and the acoustic window 40 becomes possible.

Thereby, because the amount of jelly to be interposed between the second to fifth fingers P2 to P5 and the acoustic window 40 can be reduced, the effort to apply jelly to the acoustic window 40 or the second to fifth fingers P2 to P5 and the effort to remove the jelly stuck on the acoustic window 40 or the second to fifth fingers P2 to P5 can be reduced. Further, because mixing in air that obstructs the propagation of ultrasound waves can be prevented from entering between the second to fifth fingers P2 to P5 and the acoustic window 40, deteriorating image quality of the image data of the second to fifth fingers P2 to P5 can be prevented. Furthermore, unnecessary movement of the second to fifth fingers P2 to P5 can also be suppressed, enabling the prevention of the image quality of the image data from deteriorating due to an artifact.

As described above, the ultrasound probes according to the first to sixth embodiments can include a transducer part having a plurality of transducers arranged in a two-dimensional array form, in place of a transducer part having a plurality of transducers arranged in one-dimension. In this case, a moving mechanism moves the transducer part in the direction vertical to the arrangement direction (for example, in the longitudinal direction of the holding surface) of each row of the transducers. It is also possible to transceive ultrasound waves between the transducer part and fingers contacting holding surfaces by performing an electronic scan controlled by a transceiver without providing a moving mechanism.

What is claimed is:

1. An ultrasound probe, comprising:
an acoustic window comprising more than one holding surface with a recessed surface shape that engages with a subject;
a transducer part comprising a plurality of transducers arranged in one or a plurality of rows for transceiving ultrasound waves with respect to the subject that comes into contact with the holding surface via the acoustic window; and
a moving mechanism configured to move the transducer part in a direction vertical to the arrangement direction of each row of the transducers.

2. The ultrasound probe according to claim 1, wherein
the acoustic window comprises more than two laterally curving holding surfaces with a recessed surface shape, and
the holding surfaces are formed such that the longitudinal directions thereof are parallel to each other.

3. The ultrasound probe according to claim 2, wherein
the transducers are arranged in one direction of either the lateral direction or the longitudinal direction of the holding surface, and
the moving mechanism is configured to move the transducer part in the other direction of the holding surface.

4. The ultrasound probe according to claim 1, wherein
the acoustic window comprises more than two laterally curving holding surfaces with a recessed surface shape, and
the holding surfaces are formed such that the longitudinal directions thereof are radial to each other.

5. The ultrasound probe according to claim 4, wherein the moving mechanism is configured to linearly move the transducer part in directions including directions other than the direction vertical to the longitudinal direction of the holding surface.

6. The ultrasound probe according to claim 4, wherein the moving mechanism is configured to move the transducer part in a circular arc form in the direction vertical to the longitudinal direction of the holding surface at a position of the holding surface.

7. The ultrasound probe according to claim 4, comprising
the same number of the transducer parts as the holding surfaces in which the transducers are arranged in the longitudinal direction of each holding surface,
wherein the moving mechanism is configured to move the transducer part in a circular arc form along the holding surface.

8. The ultrasound probe according to claim 4, comprising
the same number of the transducer parts as the holding surfaces in which the transducers are arranged in a circular arc form in the lateral direction of each holding surface,
wherein the moving mechanism is configured to move the transducer part in the longitudinal direction of the holding surface.

9. The ultrasound probe according to any one of claims 1 to 8, wherein
the holding surface forms a recessed surface shape laterally curving in a circular arc form, and
the radius of the circular arc is within a range of 5 to 20 mm.

10. The ultrasound probe according to claim 1, wherein
the acoustic window consists of a first acoustic window comprising more than one first holding surface with a recessed surface shape engaging with one side of the subject and a second acoustic window comprising the same number of second holding surfaces with a recessed surface shape as the first holding surfaces, the second holding surfaces engaging the other side of the subject, the other side being arranged such that the subject may be surrounded by the first acoustic window
the transducer part consists of a first transducer part comprising a plurality of first transducers arranged in one or plurality of rows for transceiving ultrasound waves with respect to the subject that comes into contact with the first holding surface via the first acoustic window and a second transducer part comprising a plurality of second transducers arranged in one or a plurality of rows for transceiving ultrasound waves with respect to the subject that comes into contact with the second holding surface via the second acoustic window, and
the moving mechanism is configured to move the first transducer part in the direction vertical to the arrangement direction of each row of the first transducers, and moves the second transducer part in the direction vertical to the arrangement direction of each row of the second transducers.

11. An ultrasound probe, comprising:
an acoustic window comprising more than one holding surface with a recessed surface shape that engages with a subject; and
a transducer part comprising a plurality of transducers arranged in a two-dimensional array form for transceiving ultrasound waves with respect to the subject that comes into contact with the holding surfaces via the acoustic window.

12. An ultrasound diagnosis apparatus, comprising:
an acoustic window comprising more than one holding surface for holding the form of a recessed surface shape that engages with a subject,
a transducer part comprising a plurality of transducers arranged in one or a plurality of rows for transceiving ultrasound waves with respect to the subject that comes into contact with the holding surfaces via the acoustic window,
a moving mechanism configured to move the transducer part in the direction vertical to the arrangement direction of each row of the transducers,
an image data generator configured to generate image data based on reception signals obtained by driving the transducer part, and
a display configured to display the image data generated by the image data generator.

13. The ultrasound diagnosis apparatus according to claim 12, wherein the display comprises the same number of display areas related to the holding surface as the holding surfaces, and displays image data generated by the image data generator in the display area related to the holding surface where ultrasound waves have been transceived by the transducer part.
